(19) United States
(12) Patent Application Publication
(10) Pub. No.: US 2022/0071856 A1
(43) Pub. Date: Mar. 10, 2022

(54) UV Protectant Formulations

(71) Applicant: B.A.I. TECHNOLOGIES, INC., CAMBRIDGE, MA (US)

(72) Inventor: Xuefei Bai, Allston, MA (US)

(21) Appl. No.: 17/318,346

(22) Filed: May 12, 2021

Related U.S. Application Data
(60) Provisional application No. 63/075,352, filed on Sep. 8, 2020.

(51) Int. Cl. A61K 8/02; A61K 8/27; A61K 8/29; A61K 8/34; A61Q 17/04

(52) U.S. Cl. CPC A61K 8/0233; A61K 8/27; A61K 8/29; A61K 8/345; B82Y 30/00; A61K 2800/651; A61K 2800/413; A61K 2800/621; A61K 2800/805; A61Q 17/04

(57) ABSTRACT

Disclosed herein are novel cosmetics or sunscreen preparations to absorb or scatter ultraviolet light and simultaneously scavenge free radicals, holes, electrons, or reactive oxygen species, and methods of preparing the same.

### RELATED APPLICATIONS

This application claims priority benefit of provisional Application Ser. No. 63/075,352 filed Sep. 8, 2020.

### TECHNICAL FIELD

The disclosure relates to cosmetics or sunscreen preparations and methods which are able to absorb or scatter ultraviolet light.

### BACKGROUND

Excessive exposure to ultraviolet (UV) radiation from the sun can cause sunburn, skin aging, and skin cancer, which is partially attributable to UV-induced reactive oxygen species (ROS). The mechanisms underlying UV-related skin aging and carcinogenesis is that UV-induced ROS can lead to DNA damage, including single- and/or double-stranded DNA breaks, base modifications, and DNA crosslinks. Sunscreens contain UV attenuators, such as chemical or physical UV filters, which can prevent damages from UV irradiation. However, it has been reported that chemical sunscreen UV filters can actually increase the UV-induced ROS once they penetrate the epidermis. US Food and Drug Administration (the FDA) has also expressed concerns over coral reef bleaching caused by organic UV filters, such as oxybenzone, octocrylene, and octinoxate. Research has revealed that chemical UV filters contribute to cellular alterations, hormonal disruptions, and carcinogenesis. Due to concerns expressed by dermatologist and other health care practitioners and researchers, the FDA has labeled p-amino-benzoic acid (PABA) and trolamine salicylate as unsafe and requested additional data for 12 other chemical UV filter ingredients, leaving no organic UV filters considered as safe and effective by the FDA to date.

On the other hand, the only two ingredients that are considered safe and effective by the FDA are physical UV filters, namely, zinc oxide and titanium dioxide. Zinc oxide and titanium dioxide are widely used as broad spectrum ultraviolet (UV) radiation filters in sunscreens. They extinct UV radiation via both scattering and non-irradiative dissipation of photon energies. The respective band gap energies of TiO₂ anatase and rutile are 3.3 eV and 2.0 eV. Therefore, photons shorter than or equal to 388 and 205 nm are able to cause the transition of an electron between the valence and conduction bands of anatase and rutile, respectively. Wurtzite ZnO, which absorbs at ˜370 nm, has a similar bandgap to anatase. However, instead of recombination, the hole-electron pairs may generate various reactive oxygen species (ROS), such as hydroxyl radicals and superoxide anions, by abstracting or donating an electron to water and oxygen molecules.

Nanodiamonds (NDs) are nanoparticles that comprise primarily sp³ carbons, which are traditionally produced by detonation. Recently developed new techniques, such as hydrothermal, ultrasound, and electrochemical syntheses, enables the manufacturing of NDs with sizes ranging from a few nanometers to sub-microns. NDs are generally considered safe for medical applications.

### SUMMARY OF THE INVENTION

Disclosed herein are novel cosmetics or sunscreen preparations to absorb or scatter ultraviolet light and simultaneously scavenge free radicals, holes, electrons, or reactive oxygen species, and methods of preparing the same.

In some aspects, the preparations comprise a core-shell structure using nanodiamonds, with abilities to physically block UV radiation and eradicate ROS generated by UV and physical filters.

One aspect may be a new particle comprising a component that absorbs or scatters ultraviolet light (Component A) and a component that scavenges free radicals, holes, electrons, or reactive oxygen species generated from a photochemical process from Component A (Component B).

In some aspects, each particle is comprised of at least one Component A domain and at least one Component B domain. The domain(s) of one component is partially or fully encapsulated by, embedded in, or attached to the domain(s) of the other component. Component A and Component B form a particle through covalent bonds, non-covalent bonds, or encapsulation of one component or multiple components within the other component. The current invention for the first time provides skin irritation/damage mitigation in sunscreen products via electron/energy transfer between a light scattering/absorbing component and a reactive species scavenger in direct contact. Direct contact between the two components permits unprecedented high efficiency in electron shuttling.

In some aspects, Component A comprises one or more of titanium dioxide, zinc oxide, organotitanium networks, boron oxide, aluminum oxide, silicon dioxide, and silsesquioxane.

In some aspects, Component B comprises one or more of redox active material and is chosen from surface modified or pristine diamond nanoparticles (nanodiamonds), carbon black particles, carbon nanotube, fullerene, graphene, graphene oxide, graphite, sulfur, styrenic polymers, poly(meth)acrylates, polyacrylamides, unsaturated polyolefins, polynorbornene derivatives, polyanilines, polyphenols, polyimines, and polyimides.

In some aspects, Components A and Component B form a core-shell structured particle, where the shell of the particle comprises Component A and the core of the particle comprises Component B.

In some aspects, the structure advantageously enables the application of particles of appropriate sizes that do not pass through the human skin corneum.

In some aspects, the structure advantageously provides practically non-expensive methods of manufacturing and applying UV protectant active ingredients to human skin.

In some aspects, the structure advantageously eliminates the uncomfortable sandy feeling of diamond particles when applied to human skin.

In some aspects, the structure advantageously requires fewer reapplications over longer periods of time.

In some aspects, the structure advantageously provides better coloring to the preparation for application to human skin.

In some aspects, the structure advantageously prevents fast oxidation of nanodiamonds.

In some aspects, a core-shell structured particle comprises titanium dioxide or zinc oxide as Component A (shell) and NDs as Component B (core), where the particle exhibits synergistic ultraviolet light attenuating properties between Component A and Component B, and unexpected enhanced elimination abilities to eliminate reactive oxygen species or other kinds of free radicals generated from a photochemical process.

In some aspects of the present invention, a cosmetic or sunscreen preparation comprises particles comprising of Component A and Component B dispensed in a carrier medium, where at least a portion of the particles are of a size no less than 80 nm, preferably no less than 100 nm. The carrier medium of the cosmetic or sunscreen preparation comprises one of physiologically compatible carrier medium.

In some aspects, core-shell particles are prepared in the following steps: Nanodiamonds are first dispersed in an organic solvent or an organic solvent mixture using an ultrasonication bath or an ultrasonication probe. The shell precursor is added to the mixture as a pure liquid or a solution in an organic solvent. The reaction mixture is refluxed. The growth of the core-shell particles is monitored using dynamic light scattering until a desired size is reached. The resulting particles are isolated by centrifuge and the solvent(s) is replaced with a low boiling point solvent or water. The product is harvested as a dry powder or an aqueous dispersion. Examples of organic solvents include but are not limited to dimethylformamide, dimethylsulfoxide, dimethylacetamide, N-methylpyrrolidone, benzene, toluene, xylene, 1,2,4-trimethylbenzene, anisole, diphenyl ether, chlorobenzene, nitrobenzene, ethyl acetate, butyl acetate, diethyl carbonate, ethylene carbonate, chloroform, carbon tetrachloride, cyclohexane, hexanes, heptanes, octanes, decanes, olefins, methanol, ethanol, 2-propanol, 1-propanol, 1-butanol, ethylene glycol, glycerin, acetic acid, propionic acid, diglyme, tetrahydrofuran, 2-methyltetrahydrofuran, dioxane, 2-butanone, acetone, methyl isobutyl ketone, pyridine, triethylamine, acetonitrile. Examples of shell precursors include but are not limit to titanium diisopropoxide bis(acetylacetonate), titanium isopropoxide, titanium oxyacetylacetonate, titanium butoxide, titanium (triethanolaminoato)isopropoxide, titanium diisopropoxidebis(2,2,6,6-tetramethyl-3,5-heptanedionate), titanium 2-ethylhexyloxide, titanium chloride, zinc acetate, zinc ethylhexanoate, zinc chloride, zinc nitrate, tetramethyl orthosilicate, tetraethyl orthosilicate, tetrachlorosilane.

In some aspects, the size of a preparation of particles is determined by the Dynamic Light Scattering method, using a measurement machine such as the Zetasizer Nano-S manufactured by Malvern, generating a size distribution by intensity graph. A size is determined to be no less than or at least a certain size in diameter when no more than 1% of the particles are smaller than the diameter according to the size distribution graph.

### DESCRIPTION OF THE DRAWINGS

FIG. 1 is an illustration of when UV irradiation is absorbed by a core-shell nanodiamond doped metal oxide particle. The UV irradiation generates an excitation, where an electron is excited from the valence band (VB) to the conduction band (CB) of the shell, leaving a hole in the CB. The hole migrates to the core-shell interface and transfers to the highest occupied molecular orbital (HOMO) of nanodiamond. The nanodiamond core scavenges the hole either by an internal oxidation process or by harvesting the electron in CB of the shell.

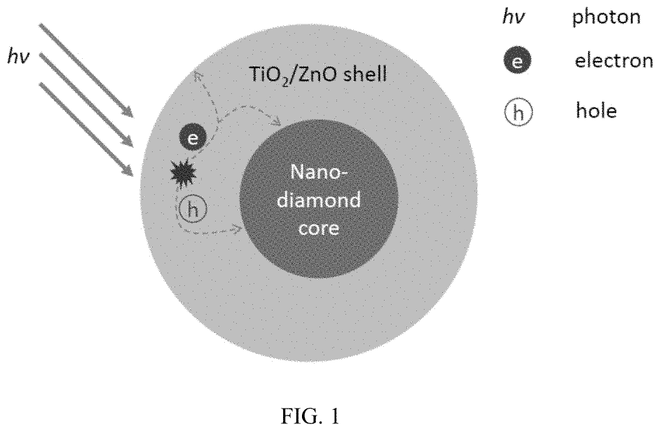

FIG. 2 illustrates the process where the hole migrates to the core-shell interface and transfers to the HOMO of nanodiamond.

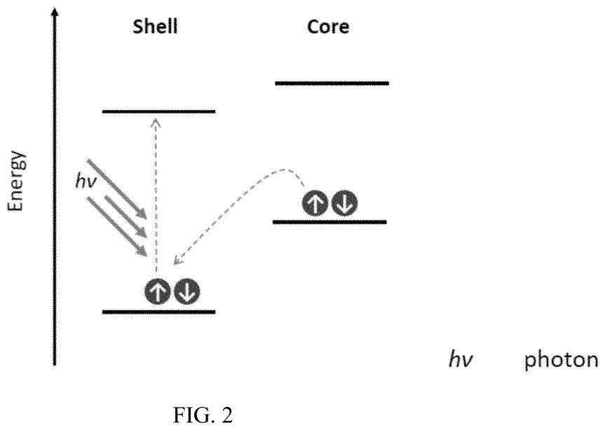

FIG. 3 illustrates the intensity-weighted hydrodynamic size distribution of core-shell nanodiamond-ZnO particles synthesized in Example 1, with a Z-average around 290 nm.

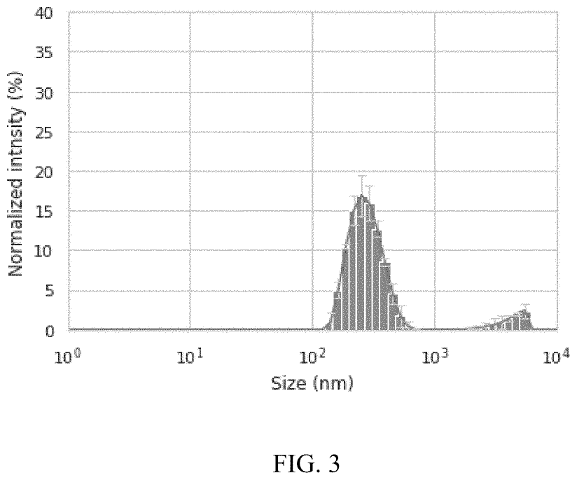

FIG. 4 illustrates the intensity-weighted hydrodynamic size distribution of core-shell nanodiamond-TiO₂ particles synthesized in Example 2, with a Z-average around 143 nm.

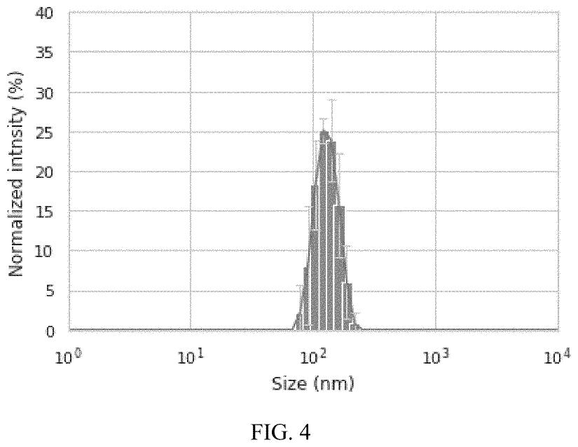

FIG. 5 illustrates the intensity-weighted hydrodynamic size distribution of core-shell nanodiamond-ZnO particles synthesized in Example 3, with a Z-average around 149 nm.

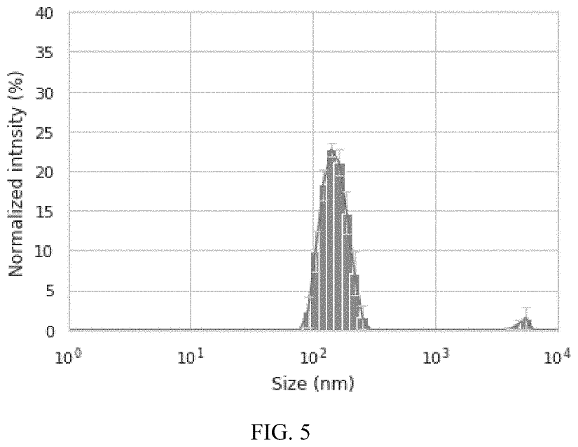

FIG. 6 illustrates the intensity-weighted hydrodynamic size distribution of core-shell nanodiamond-TiO₂ particles synthesized in Example 4, with a Z-average around 1558 nm.

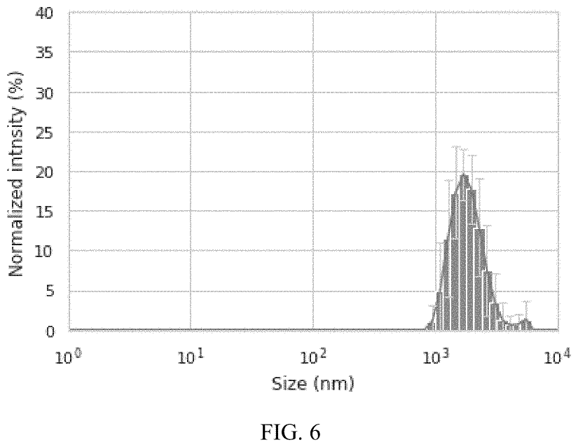

FIG. 7 illustrates the intensity-weighted hydrodynamic size distribution of core-shell nanodiamond-ZnO particles synthesized in Example 5, with a Z-average around 1386 nm.

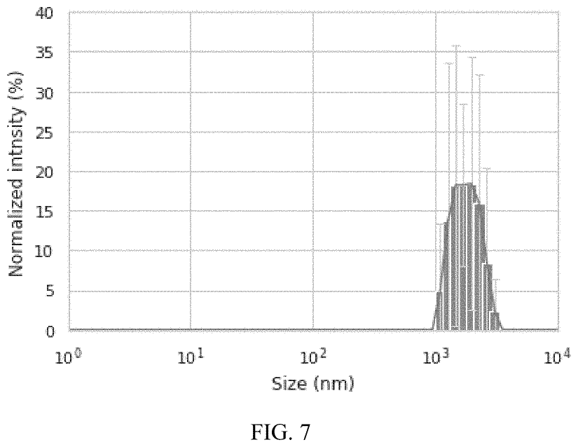

FIG. 8 illustrates that the core-shell nanodiamond-ZnO particles exhibit significantly better ultraviolet (UV) blocking capabilities compared to ZnO. ZnO USP and ZnO Micronized are ZnO currently used in sunscreen products in the US, purchased through makingcosmetics.com. “BAI0002 S” refers to core-shell nanodiamond-ZnO particles with Z-average around 150 nm. “BAI0002 B” refers to core-shell nanodiamond-ZnO particles with Z-average around 300 nm. The UV-Vis absorption spectrum is measured using Nanodrop.

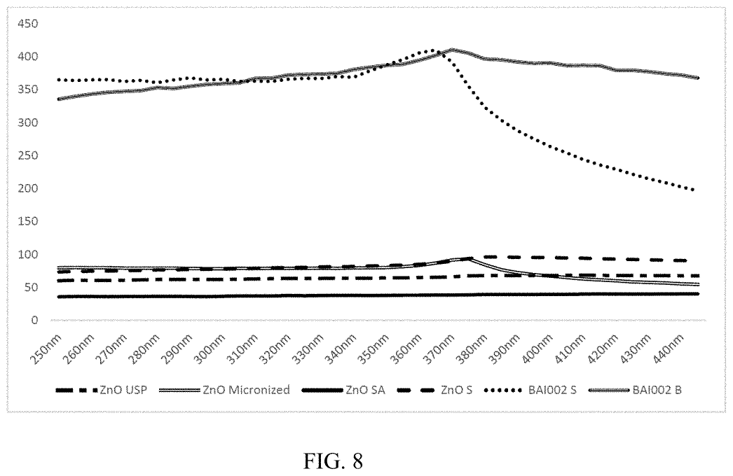

FIG. 9 illustrates that at the concentration of 1 mg/ml, core-shell nanodiamond-ZnO particles exhibit much higher capabilities to eradicate ROS compares to ZnO (Control S). “BAI0002 S” refers to core-shell nanodiamond-ZnO particles with Z-average around 150 nm.

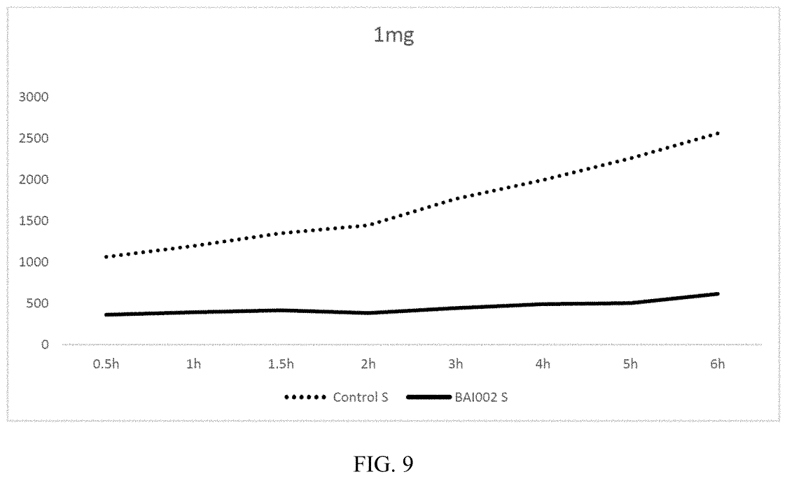

FIG. 10 illustrates that at the concentration of 10 mg/ml, core-shell nanodiamond-ZnO particles exhibit much higher capabilities to eradicate ROS compares to ZnO (Control S). “BAI0002 S” refers to core-shell nanodiamond-ZnO particles with Z-average around 150 nm.

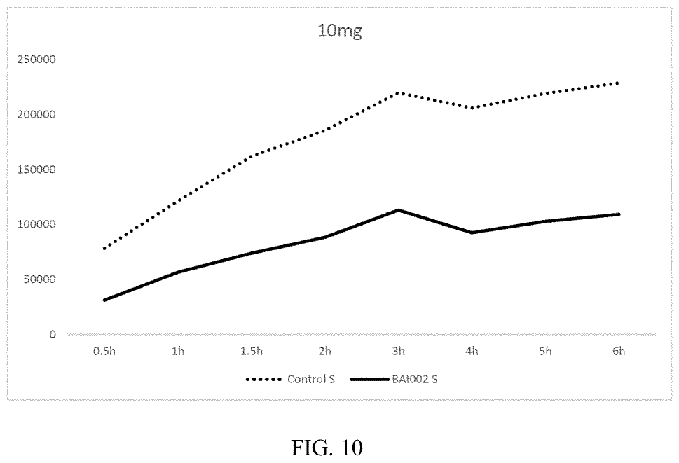

FIG. 11 shows that core-shell nanodiamond-ZnO particles are nontoxic and nonirritating based on the Modified Epiderm Skin Irritation Test (SIT). The columns represent the relative viability of cells in the Modified Epiderm SIT, from left to right, the cells were treated with 1) negative control (NC); 2) positive control (PC); 3) 25% commercial ZnO suspension (Commerical ZnO 25%); 4) 5% suspension of core-shell nanodiamond-ZnO particles with Z-average around 150 nm (ZnO ND small 5%); 5) 5% suspension of core-shell nanodiamond-ZnO particles with Z-average around 300 nm (ZnO ND big 5%); 6) 25% solution of core-shell nanodiamond-ZnO particles with Z-average around 150 nm (DEG ZnO ND small 25%); 7) 25% solution of core-shell nanodiamond-ZnO particles with Z-average around 150 nm (ZnO ND small 25%); and 8) 25% solution of core-shell nanodiamond-ZnO particles with Z-average around 300 nm (ZnO ND big 25%). All the suspensions are prepared in water.

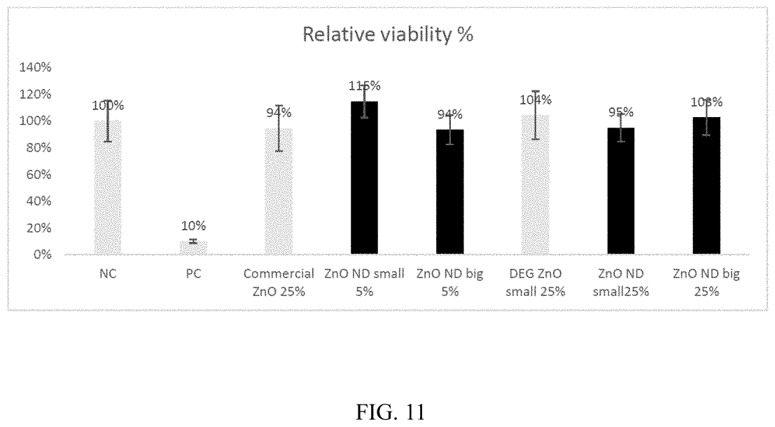

FIG. 12 illustrates the shapes of the claimed particles can be, but are not limited to, sphere, distorted sphere, cylinder, polyhedron, or platelet with the largest dimension at least 80 nanometers.

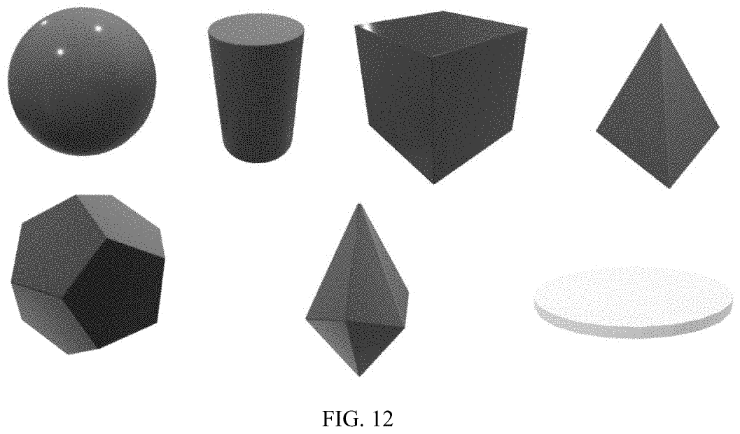

FIG. 13 illustrates that each particle comprises at least one Component A domain and at least one Component B domain. The Component B domain(s) is partially or fully encapsulated by, embedded in, or attached to the Component A domain(s).

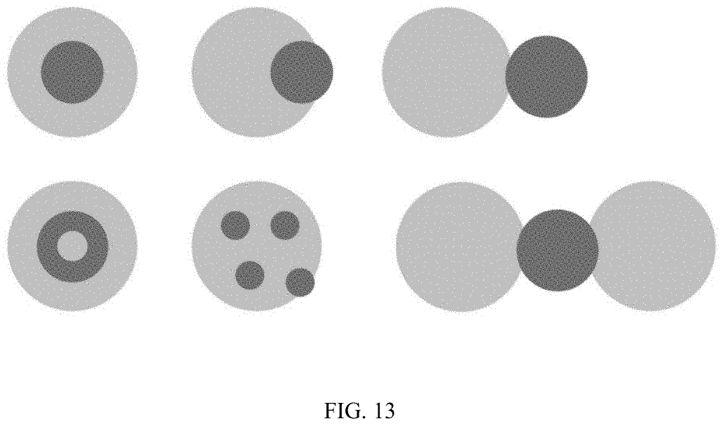

### DETAILED DESCRIPTION

I. Definitions

To facilitate an understanding of the present disclosure, a number of terms and phrases are defined below:

The term “nanodiamond” refers to particles with diameters between 1-1000 nanometers comprising predominantly sp3 carbons.

The term “hole electron pair”, also termed “exciton”, refers to a pair of hole and electron attracted to each other by Coulombic force.

The term “hole” refers to the lack of an electron where one could exist.

The term “core-shell” or “core-shell structure” refers to a structure of a particle where a first component is partially or fully encapsulated by or embedded in a second component, such that no more than half of the first component's surface area is exposed. In this case, the first component is a core, and the second component is a shell. The core and the shell may form a particle through covalent bonds, non-covalent bonds, or encapsulation.

The term “Z average” is the intensity weighted mean hydrodynamic size of the ensemble collection of particles measured by dynamic light scattering (DLS). The Z average is derived from a Cumulants analysis of the measured correlation curve, wherein a single particle size is assumed and a single exponential fit is applied to the autocorrelation function.

II. The Invention

The present disclosure is directed to cosmetics or sunscreen preparations and methods which are able to absorb or scatter ultraviolet light and simultaneously scavenge free radicals, holes, electrons, or reactive oxygen species.

In some aspects, each particle is comprised of at least one Component A domain and at least one Component B domain. The domain(s) of one component is partially or fully encapsulated by, embedded in, or attached to the domain(s) of the other component. Component A and Component B form a particle through covalent bonds, non-covalent bonds or encapsulation of one component or multiple components within the other component. The current invention provides skin irritation/damage mitigation in sunscreen products via electron/energy transfer between a light scattering/absorbing component and a reactive species scavenger in direct contact. Direct contact between the two components permits high efficiency in electron shuttling.

In some aspects, Component A comprises one or more of titanium dioxide, zinc oxide, organotitanium networks, boron oxide, aluminum oxide, silicon dioxide, and silsesquioxane.

In some aspects, Component B is redox active material and comprises one or more of surface modified or pristine diamond nanoparticles (nanodiamonds), carbon black particles, carbon nanotube, fullerene, graphene, graphene oxide, graphite, sulfur, styrenic polymers, poly(meth)acrylates, polyacrylamides, unsaturated polyolefins, polynorbornene derivatives, polyanilines, polyphenols, polyimines, and polyimides.

In some aspects, Components A and Component B form a core-shell structured particle, where the shell of the particle comprises Component A and the core of the particle comprises Component B.

In some aspects, the structure advantageously enables application of particles of appropriate sizes that do not pass through human skin corneum because the particles are of a size of at least 100 nm.

In some aspects, the structure provides practically non-expensive methods of manufacturing and applying UV protectant active ingredients to human skin. In some aspects, the structure eliminates the uncomfortable sandy feeling of diamond particles when applied to human skin. In some aspects, the structure requires fewer reapplications over longer periods of time. In some aspects, the structure provides better coloring to the preparation for application to human skin. In some aspects, the structure prevents fast oxidation of nanodiamonds.

In some aspects, a core-shell structured particle comprises titanium dioxide or zinc oxide as Component A (shell) and nanodiamonds as Component B (core), where the particle exhibits synergistic ultraviolet light attenuating properties between Component A and Component B, and enhanced elimination abilities to eliminate reactive oxygen species or other kinds of free radicals generated from a photochemical process.

In some aspects of the present invention, a cosmetic or sunscreen preparation comprises particles comprising of Component A and Component B dispensed in a carrier medium, where at least a portion of the particles are of a size no less than 80 nm, preferably no less than 100 nm. The carrier medium of the cosmetic or sunscreen preparation comprises one of physiologically compatible carrier medium.

In some aspects, the core-shell particles are prepared in the following steps: nanodiamonds are first dispersed in an organic solvent or an organic solvent mixture using an ultrasonication bath or an ultrasonication probe. The shell precursor is added to the mixture as a pure liquid or a solution in an organic solvent. The reaction mixture is refluxed. Growth of the core-shell particles is monitored using dynamic light scattering until a desired size is reached. The resulting particles is isolated by centrifuge and the solvent(s) is replaced with a low boiling point solvent or water. The product is harvested as a dry powder or an aqueous dispersion. Examples of organic solvents include but are not limited to dimethylformamide, dimethylsulfoxide, dimethylacetamide, N-methylpyrrolidone, benzene, toluene, xylene, 1,2,4-trimethylbenzene, anisole, diphenyl ether, chlorobenzene, nitrobenzene, ethyl acetate, butyl acetate, diethyl carbonate, ethylene carbonate, chloroform, carbon tetrachloride, cyclohexane, hexanes, heptanes, octanes, decanes, olefins, methanol, ethanol, 2-propanol, 1-propanol, 1-butanol, ethylene glycol, glycerin, acetic acid, propionic acid, diglyme, tetrahydrofuran, 2-methyltetrahydrofuran, dioxane, 2-butanone, acetone, methyl isobutyl ketone, pyridine, triethylamine, acetonitrile. Examples of shell precursors include but are not limited to titanium diisopropoxide bis(acetylacetonate), titanium isopropoxide, titanium oxyacetylacetonate, titanium butoxide, titanium (triethanolaminoato)isopropoxide, titanium diisopropoxidebis(2,2,6,6-tetramethyl-3,5-heptanedionate), titanium 2-ethylhexyloxide, titanium chloride, zinc acetate, zinc ethylhexanoate, zinc chloride, zinc nitrate, tetramethyl orthosilicate, tetraethyl orthosilicate, tetrachlorosilane.

As discussed herein, the size of a preparation of particles is determined by the Dynamic Light Scattering method, using a measurement machine such as the Zetasizer Nano-S manufactured by Malvern, generating a size distribution by intensity graph. A size is determined to be no less than or at least a certain size in diameter when no more than 1% of the particles are smaller than the diameter according to the size distribution graph.

### A. GENERAL PREPARATION OF CORE-SHELL NANODIAMOND DOPED METAL OXIDE PARTICLES

In some aspects, nanodiamonds are dispersed by ultrasonication at a weight fraction of 0.01%-1% in a high boiling point polar solvent. Then a precursor is added to a weight fraction of 1-20% to this mixture. Once the precursor was dissolved, the mixture is heated at a rate of 1-10° C./min to a temperature between 150-200° C. while vigorously stirred.

In some aspects, the mixture is held at the temperature for 10-30 minutes or until the size distribution of the particles is entirely above 100 nm. After the reaction, the mixture was cooled to room temperature.

In some aspects, the solids were isolated by centrifuge and washed with a low to moderate boiling point solvent or water in multiple cycles.

In some aspects, the product was air-dried and baked at 120° C. to remove any residual solvents.

In some aspects, the size of the particles was measured with a Malvern Zetasizer Nano S particle analyzer.

### B. EXAMPLES

The following examples are provided in order to demonstrate and further illustrate certain aspects of the present disclosure and are not to be construed as limiting the scope thereof.

As described herein, the ZnO or core-shell nanodiamond-ZnO particles are dispersed in distilled water to final concentration of 5 mg/ml and the UV-Vis absorption spectrum is measured using Nanodrop.

As described herein, intracellular production of ROS was measured using 2,7-dichlorofluorencein diacetate (DCFH-DA) passively enters the cell, where it reacts with ROS to form the highly fluorescent compound dichlorofluorescein (DCF). 4.9 mg/mL DCFH-DA is solved in DMSO, then diluted for 100 times with 1×PBS to make working solution. Nanodiamond-ZnO particle suspensions and ZnO suspensions are made to final concentrations of 10 mg/mL or 1 mg/mL in the premade working solution. 200 uL of either nanodiamond-ZnO particle suspensions or ZnO suspensions were added to each well of the 96-well plate in triplicates. The plate was then exposed under natural light at room temperature. Fluorescence was measured at 0.5 h, 1 h, 1.5 h, 2 h, 3 h, 4 h, 5 h, and 6 h using plate reader, under 485 nm excitation and 520 nm emission.

As described herein, the Modified Epiderm SIT, MTT solution is prepared using the MatTek MTT toxicology kit (Part #MTT-100). A MTT plate is prepared by pipetting 300 μl of the MTT solution into the appropriate number of wells of the 24-well plate. After exposing EpiDerm samples to ZnO, nanodiamond-ZnO particles, positive control and negative control, liquid remaining atop the EpiDerm tissues is decanted. Inserts with EpiDerm tissues are removed and rinsed with PBS for one or more times. Excess liquid is shaken off prior to placing the EpiDerm sample in the MTT containing 24-well plate while making sure that no air bubbles are trapped underneath the cell culture insert. The EpiDerm samples in the 24-well plate are then return to the incubator for 3 hours. After the 3-hour MTT incubation period is complete, each insert is removed individually and the bottom is gently blotted with a Kim Wipe. The inserts are then placed into the pre-labeled 24-well extraction plate and immersed with 2.0 ml of the extractant solution per well to completely cover the EpiDerm sample. The extraction plates are covered to reduce evaporation of extractant and then incubated in the dark at room temperature overnight. Then, the liquid within each insert was decanted back into their corresponding original wells. The inserts were discarded. The extractant solution were thoroughly mixed and transferred in 200 μL aliquots with triplicates. The optical density of the extracted samples was determined at 570 nm using 200 μl of extractant as a blank and the viability was determined using the following equation.

% viability=100×[OD(sample)/OD(negative control)]

### Example 1

5 mg of nanodiamonds of a size between 3-10 nm are dispersed in 50 ml of diethylene glycol (DEG) by ultrasonication. 1.98 g (9 mmol) of zinc acetate dihydrate is then added to this mixture. The mixture is heated to 180 degrees Celsius at a rate of 6 degrees per minute while vigorously stirring and held at 180 degrees for 15 minutes. After the reaction, the mixture is cooled to room temperature. The solids are isolated by centrifuge and then washed with water in 3 cycles. The sample is dried in air overnight before baked at 120 degrees to remove any residual solvents.

### Example 2

5 mg of nanodiamonds of a size between 3-10 nm are dispersed in 50 ml of diethylene glycol (DEG) by ultrasonication. 2 g (16 mmol) of tetrabutyl titantate is then added to this mixture. The mixture is heated to 180 degrees Celsius at a rate of 6 degrees per minute while vigorously stirring and held at 180 degrees for 15 minutes. After the reaction, the mixture is cooled to room temperature. The solids are isolated by centrifuge and then washed with water in 3 cycles. The sample is dried in air overnight before baked at 120 degrees to remove any residual solvents.

### Example 3

5 mg of nanodiamonds of a size between 3-10 nm are dispersed in 50 ml of diethylene glycol (DEG) by ultrasonication. 1.98 g (9 mmol) of zinc acetate dihydrate is then added to this mixture. The mixture is heated to 180 degrees Celsius at a rate of 6 degrees per minute while vigorously stirring and held at 180 degrees for 15 minutes. After the reaction, the mixture is cooled to room temperature. The solids are isolated by centrifuge and then washed with ethanol in 3 cycles. The sample is dried in air overnight before baked at 120 degrees to remove any residual solvents.

### Example 4

2 mg of nanodiamonds of a size between 3-10 nm is dispersed in 48 mL of N,N-dimethylformamide (DMF) using a ultrasonication bath in a 100 mL round bottom flask. 2 mL of tetrabutyl orthotitanate (Ti(OBu)₄) is then added to the solution. A waterless condenser is installed on the top of the flask and slowly bring the reaction to reflux (−155 degrees Celsius). The reaction is then cooled down to ˜50 degrees Celsius during which time samples are taken for dynamic light scattering every 2 hours. The reaction is stopped when the entire size distribution of the particles is above 100 nm. The nanoparticles are collected by centrifuge and then washed with acetone to remove DMF and unreacted Ti(OBu)₄.

### Example 5

2 mg of nanodiamonds of a size between 3-10 nm is dispersed in 48 mL of N,N-dimethylformamide (DMF) using a ultrasonication bath in a 100 mL round bottom flask. 2 mL of tetrabutyl orthotitanate (Zn(OAc)₂) is then added to the solution. A waterless condenser is installed on the top of the flask and slowly bring the reaction to reflux (−155 degrees Celsius). The reaction is then cooled down to ˜50 degrees Celsius during which time samples are taken for dynamic light scattering every 2 hours. The reaction is stopped when the entire size distribution of the particles is above 100 nm. The nanoparticles are collected by centrifuge and then washed with acetone to remove DMF and unreacted Zn(OAc)₂.

We claim:

1. A composition comprising a first component and a second component, wherein:
the first component comprises one or more of nanodiamonds, carbon black particles, carbon nanotube, fullerene, graphene, graphene oxide, graphite, sulfur, styrenic polymers, poly(meth)acrylates, polyacrylamides, unsaturated polyolefins, polynorbornene derivatives, polyanilines, polyphenols, polyimines, and polyimides;
the second component comprises one or more of titanium dioxide, zinc oxide, organotitanium networks, boron oxide, aluminum oxide, silicon dioxide, and silsesquioxane; and
the first component and the second component form particles of a core-shell structure, wherein the first component is a core and the second component is a shell.

2. A composition according to claim 1, wherein the first component comprises nanodiamonds.

3. A composition according to claim 2, wherein the size of the composition is at least 80 nm.

4. A composition according to claim 3, wherein the size of the composition is at least 100 nm.

5. A composition according to claim 2, wherein the second component comprises titanium dioxide or zinc oxide.

6. A composition according to claim 3, wherein the second component comprises titanium dioxide or zinc oxide.

7. A composition according to claim 4, wherein the second component comprises titanium dioxide or zinc oxide.

8. A method of preparing particles of a core-shell structure, comprising:
dispersing nanodiamonds in an organic solvent;
adding a shell precursor, resulting in a reaction mixture; and
heating the reaction mixture, resulting in core-shell particles, until the core-shell particles reach a size.

9. A method according to claim 8, wherein the organic solvent comprises one or more of dimethylformamide, dimethylsulfoxide, dimethylacetamide, N-methylpyrrolidone, benzene, toluene, xylene, 1,2,4-trimethylbenzene, anisole, diphenyl ether, chlorobenzene, nitrobenzene, ethyl acetate, butyl acetate, diethyl carbonate, ethylene carbonate, chloroform, carbon tetrachloride, cyclohexane, hexanes, heptanes, octanes, decanes, olefins, methanol, ethanol, 2-propanol, 1-propanol, 1-butanol, ethylene glycol, glycerin, acetic acid, propionic acid, diglyme, tetrahydrofuran, 2-methyltetrahydrofuran, dioxane, 2-butanone, acetone, methyl isobutyl ketone, pyridine, triethylamine, and acetonitrile.

10. A method according to claim 9, wherein the shell precursor comprise one or more of titanium diisopropoxide bis(acetylacetonate), titanium isopropoxide, titanium oxyacetylacetonate, titanium butoxide, titanium (triethanolaminoato)isopropoxide, titanium diisopropoxidebis(2,2,6,6-tetramethyl-3,5-heptanedionate), titanium 2-ethylhexyloxide, titanium chloride, zinc acetate, zinc ethylhexanoate, zinc chloride, zinc nitrate, tetramethyl orthosilicate, tetraethyl orthosilicate, and tetrachlorosilane.

11. A method according to claim 10, wherein the nanodiamonds are dispersed in the organic solvent at a fraction of 0.01% to 1% by weight.

12. A method according to claim 11, wherein the shell precursor is added in the reaction mixture at a fraction of 1% to 20% by weight.

13. A method according to claim 12, wherein the reaction mixture is heated to a temperature between 150° C. and 200° C. at a rate of 1° C. per minute to 10° C. per minute.

14. A method according to claim 13, wherein the size is 100 nm.

15. A cosmetic or sunscreen preparation comprising particles of a core-shell structure, wherein a first component is a core and a second component is a shell, and wherein the first component comprises nanodiamonds, and wherein the second component comprises titanium dioxide or zinc oxide.
